(19) United States
(12) Patent Application Publication
(10) Pub. No.: US 2024/0165085 A1
(43) Pub. Date: May 23, 2024

(54) ORAL FELINE FEED AND METHODS FOR CONTROLLING TICK INFESTATIONS IN A FELINE

(71) Applicant: In the Bowl Animal Health, Inc., Nashville, TN (US)

(72) Inventors: Robin S. Readnour, Nashville, TN (US); Kevin E. Willard, Knightstown, IN (US); Joseph R. Winkle, Carmel, IN (US)

(21) Appl. No.: 18/543,614

(22) Filed: Dec. 18, 2023

Related U.S. Application Data
(63) Continuation of application No. PCT/US22/34746, filed on Jun. 23, 2022.
(60) Provisional application No. 63/214,999, filed on Jun. 25, 2021.

(51) Int. Cl. A61K 31/422; A23L 33/00; A61P 33/14

(52) U.S. Cl. CPC A61K 31/422; A23L 33/40; A61P 33/14

(57) ABSTRACT

An oral feline feed and a method of controlling ticks in a feline in need thereof by orally administering to the feline a daily feed comprising an effective amount of a isoxazoline for an effective time to thereby cause the amount of isoxazoline in the feline's blood to rise to and maintain at a therapeutically effective level for controlling ticks.

### RELATED APPLICATIONS

This application is a continuation of International Application Serial No. PCT/US2022/034746, filed Jun. 23, 2022, which claims priority to U.S. Patent Application Ser. No. 63/214,999, filed Jun. 25, 2021, the entire disclosures of both of which are hereby incorporated herein by reference.

### TECHNICAL FIELD

The teachings of this disclosure generally relate to an isoxazoline, a feline feed that includes the isoxazoline and a method of administering the isoxazoline in a feed to control tick infestations in felines.

### BACKGROUND AND SUMMARY

In addition to being a nuisance, ticks create significant health risks to felines and their owners. Ticks are vectors of a number of different pathogenic agents. Examples of diseases which are caused by ticks include borreliosis (Lyme disease caused by Borrelia burgdorferi), babesiosis (or piroplasmosis caused by Babesia microti) and rickettsiosis (Rocky Mountain spotted fever). Ticks also release toxins, which can cause inflammation or paralysis in the host.

A recent survey of ticks infesting cats in the United States found that cats are at risk of tick infestation throughout the year, not just in the summer months. M. Saleh et al., Show Us Your Ticks: A Survey of Ticks Infesting Dogs and Cats across the USA, Parasites & Vectors, December 2019, pgs. 595-606, vol. 12. The study further found evidence of tick infestations in 49 of 50 states and evidence of tick species spreading to new geographical regions. Id. One of the recommended ways to control human risk from ticks is to control the risk of infestation in cats.

Treatments currently available for controlling tick infestations in felines achieve varying degrees of success. Many treatments involve chemicals applied to indoor and outdoor surfaces, as well as to the feline. The chemicals used include a variety of acaricidal products that contain certain macrocyclic lactones, fiproles and pyrethroids. These compounds often have toxic side effects that are a problem for both the feline and its owner. In addition, there is evidence that the use of these chemicals may be ineffective due to acaricide resistance and treatment deficiencies.

Topical treatments, which includes the use of collars, are a well-known method for controlling tick infestations in felines. While there are numerous ways to deliver these therapeutic agents to the coats and skins of felines, many of these methods are either ineffective and/or present safety risks to the feline or user during or after the dispensing activity. More particularly, because a physical connection must be achieved between the applicator tip and the drug delivery device when the applicator tip is installed thereon, there is inherently a risk that the connection will be inadequate, thereby permitting some of the therapeutic agent to leak out of the device and into physical contact with the user. For example, in the case of uncooperative felines, it may be difficult to maneuver the dispenser with one hand and maintain the feline in place with the other hand, resulting in some, if not all, of the substance being spilled on the floor or on the person applying it instead of reaching the feline's skin. Not only is this leakage wasteful and messy, it also places the user at a heightened risk of suffering from a skin irritation or other such health concern, particularly if the user comes into direct contact with the agent.

Oral treatments are also available. However, to be effective, the feline owner must administer a treatment once every 30-90 days, for example. The extended time between treatments creates compliance issues when owners forget to administer doses.

Despite the availability of effective treatments, a recent study by The Harris Poll found that 33% of pet owners do not routinely protect their pets against ticks at all. Another study found that pet owners purchased, on average, only 4 months of tick prevention products per year per pet, despite being told that pets needed to be given tick prevention treatments year-round. Thus, there continues to be a need for relatively safe, effective agents for controlling tick infestations on felines that is easier for owners to remember to use.

Surprisingly, it has been discovered by the inventors that isoxazolines can provide improved control over tick infestations in felines when orally administered in smaller, more frequent/chronic doses. The administration is discussed below as being combined with feed. However, it is also contemplated that the isoxazoline may be administered by itself or in a dosage form other than feed, such as a chew, tablet, liquid, gel or other suitable form for oral administration. Advantageously, by using smaller, more frequent doses, less total isoxazoline is required over the same time period to control tick infestations. Assume, for example, that 24 mg of isoxazoline/kg of feline body weight is needed for a single dose in a 30-day (1-month) period according to the prior art approach to reach and maintain a therapeutically effective concentration of isoxazoline in the feline's blood for continued tick control. With the inventive approach of smaller and more frequent doses, as little as 0.1-0.72 mg of isoxazoline/kg of feline body weight may be needed per day, or 3-21.6 of isoxazoline/kg of feline body weight cumulative over the same 30-day period.

Advantageously, the total amount of isoxazoline required for a therapeutically effective once-monthly dose can be reduced by 10-87.5% by converting to daily administration. However, from a practical perspective, at least two problems arise: (1) creating a homogenous feed; and (2) analytical control testing for a very small dose of isoxazoline may be difficult to accomplish. The analytical matrix from feeds can be quite complex and difficult to assay. Assays will be in the parts per million to billion range for some needed dose and feed concentrations. Thus, it is possible that one of skill in the art may opt to increase the daily dose such that the total of the daily doses over the course of one month equals the prior art once-monthly dose or is even higher, for example, 200% of the prior art once-monthly dose. This may be done to help ensure homogeneity as well as increase assay accuracy and decrease analytical variability when administering the dose as part of a daily feed.

The method and composition taught herein have the further advantage of encouraging compliance because the smaller doses of an isoxazoline can be incorporated into a feed. Since owners naturally follow a daily feeding regimen in any event, this makes it less likely that owners will forget or neglect to administer the treatment. Thus, this disclosure provides a method for prolonged control of ticks in a safer and more effective manner than that achieved with previously known treatment methodologies. All the owner need remember is to feed their pet as they normally would.

Further, the bioavailability of certain isoxazolines can be improved by administering them with feed. Thus, this disclosure provides a method for prolonged control of ticks in a safer and more effective and convenient manner than that achieved with previously known treatments.

Isoxazolines are a class of five-membered heterocyclic chemical compounds, containing one atom each of oxygen and nitrogen which are located adjacent to one another.

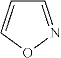

Isoxazolines are all derivatives of isoxazole. They are structural isomers of the more common oxazolines and exist in three different isomers depending on the location of the double bond.

Isoxazoline derivatives are known. For example, WO2007/105814, WO2008/122375, and WO2009/035004 disclose certain alkylene linked amides. WO2010/032437 discloses that the benzyl amide can be moved to the position ortho to the isoxazoline. WO2007/075459 discloses phenyl isoxazolines substituted with 5- to 6-membered heterocycles, and WO2010/084067 and WO2010/025998 disclose phenyl isoxazolines substituted with 10- to 11-membered fused aryl and heteroaryls. Chiral processes for manufacturing isoxazolines are disclosed in WO2011/104089 and WO2009/063910.

A number of isoxazolines compounds are known, including but not limited to 4-(5-methyl-5-substituted pyrrolyl-4,5-dihydroisoxazole-3-yl) benzoic acid amide derivatives; 4-(5-substituted carbamoylmethyl-4,5-dihydroisoxazole-3-yl) benzoic acid amide derivatives: 3-(5-substituted carbamoylmethyl-5-substituted alkyl-4,5-dihydroisoxazole-3-yl) benzoic acid amide derivatives; 4-(5-substituted carbamoylmethyl-4,5-dihydroisoxazole-3-yl) benzamidine derivatives: 4-(5-substituted-5-substituted aryl-4,5-dihydroisoxazole-3-yl)benzoic acid amide compounds; 3-4-substituted phenyl)-4,5-dihydroisoxazole derivatives; 5-substituted alkyl-3,5-bis substituted phenyl-4,5-dihydroisoxazole derivatives; 3-alkoxyphenyl-5-substituted-5-phenyl-4,5-dihydroisoxazole derivatives; 3-alkoxyphenyl-5-substituted alkyl-5-substituted carbamoyl-4,5-dihydroisoxazole derivatives; 3-(4-halophenyl)-5-substituted-5-substituted phenyl-4,5-dihydroisoxazole derivatives; 3-(4-nitrophenyl)-5-substituted-5-substituted phenyl-4,5-dihydroisoxazole derivatives: 4-hydroxyiminomethyl benzoic acid amide derivatives; 4-hydroxyiminomethyl-N,N-dimethyl benzoic acid amide; 4-hydroxyiminomethyl benzoyl piperidine derivatives: 4-hydroxyiminomethyl-N-bicycloalkyl benzoic acid amide derivatives; 6-(hydroxyiminomethyl) pyridine-2-carboxamide derivatives; haloalkenylbenzene derivatives, such as substituted 3,3,3-trifluoro-2-propenylbenzene derivatives; 4-(isoxazolinyl)-benzamides, such as substituted 4-(5-(halomethyl)-5-phenyl-isoxazolin-3-yl)-benzamides; 4-(isoxazolinyl)-benzothioamides, such as substituted 4-(5-(halomethyl)-5-phenyl-isoxazolin-3-yl)-benzothioamides; dihydroisoxazole compounds; and spirocyclic substituted isoxazolines.

Isoxazolines of particular interest for controlling tick infestations in felines are afoxolaner (chemical names: (a) 1-Naphthalenecarboxamide, 4-[5-[3-chloro-5-(trifluoromethyl)phenyl]-4,5-dihydro-5-(trifluoromethyl)-3-isoxazolyl]-N-[2-oxo-2-[(2,2,2-trifluoroethyl)amino]ethyl]-; or (b) 4-{5-[3-chloro-5-(trifluoromethyl)phenyl]-5-(trifluoromethyl)-4,5-dihydroisoxazol-3-yl}-N-{2-oxo-2-[(2,2,2-trifluoroethyl)amino]ethyl}naphthalene-1-carboxamide), fluralaner (chemical names: (a) Benzamide, 4-[5-(3,5-dichlorophenyl)-4,5-dihydro-5-(trifluoromethyl)-3-isoxazolyl]-2-methyl-N-[2-oxo-2-[(2,2,2-trifluoroethyl)amino]ethyl]-; or (b) 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide), sarolaner (chemical names: (a) Ethanone, 1-[5′-[(5S)-5-(3,5-dichloro-4-fluorophenyl)-4,5-dihydro-5-(trifluoromethyl)-3-isoxazolyl]spiro[azetidine-3,1′(3′H)-isobenzofuran]-1-yl]-2-(methylsulfonyl)-; or (b) 1-{5′-[(5S)-5-(3,5-dichloro-4-fluorophenyl)-5-(trifluoromethyl)-4,5-dihydroisoxazol-3-yl]-3′-H-spiro[azetidine-3,1′-[2]benzofuran]-1-yl}-2-(methylsulfonyl)ethanone), lotilaner (chemical names: (a) 2-Thiophenecarboxamide, 5-[(5S)-4,5-dihydro-5-(3,4,5-trichlorophenyl)-5-(trifluoromethyl)-3-isoxazolyl]-3-methyl-N-[2-oxo-2-[(2,2,2-trifluoroethyl)amino]ethyl]-; or (b) 3-methyl-N-{2-oxo-2-[(2,2,2-trifluoroethyl)amino]ethyl}-5-[(5S)-5-(3,4,5-trichlorophenyl)-5-(trifluoromethyl)-4,5-dihydro-1,2-oxazol-3-yl]thiophene-2-carboxamide), esafoxolaner (chemical names: (a) 1-Naphthalenecarboxamide, 4-[(5S)-5-[3-chloro-5-(trifluoromethyl)phenyl]-4,5-dihydro-5-(trifluoromethyl)-3-isoxazolyl]-N-[2-oxo-2-[(2,2,2-trifluoroethyl)amino]ethyl]-; or (b) (S)-4-(5-(3-chloro-5-(trifluoromethyl)phenyl)-5-(trifluoromethyl)-4,5-dihydroisoxazol-3-yl)-N-(2-oxo-2-((2,2,2-trifluoroethyl)amino)ethyl)-1-naphthamide), tigolaner (chemical names: (a) Benzamide, 2-chloro-N-(1-cyanocyclopropyl)-5-[1′-methyl-3′-(1,1,2,2,2-pentafluoroethyl)-4′-(trifluoromethyl)[1,5′-bi-1H-pyrazol]-4-yl]-; or (b) 2-chloro-N-(1-cyanocyclopropyl)-5-[2′-methyl-5′-(pentafluoroethyl)-4′-(trifluoromethyl)-2′H-[1,3′-bipyrazol]-4-yl]benzamide), umifoxolaner (chemical names: (a) 1-Naphthalenecarboxamide, 4-[(5S)-5-[3-chloro-4-fluoro-5-(trifluoromethyl)phenyl]-4,5-dihydro-5-(trifluoromethyl)-3-isoxazolyl]-N-[2-oxo-2-[(2,2,2-trifluoroethyl)amino]ethyl]-; or (b) 4-{(5S)-5-[3-chloro-4-fluoro-5-(trifluoromethyl)phenyl]-5-(trifluoromethyl)-4,5-dihydroisoxazol-3-yl}-N-{2-oxo-2-[(2,2,2-trifluoroethyl)amino]ethyl}naphthalene-1-carboxamide), modoflaner (chemical name: 6-fluoro-N-(2-fluoro-3-{[4-(heptafluoropropan-2-yl)-2-iodo-6-(trifluoromethyl)phenyl]carbamoyl}phenyl)pyridine-3-carboxamide) and mivorilaner (chemical names: (a) 4H-Cyclopenta[c]thiophene-1-carboxamide, 3-[(5S)-5-(3,5-dichloro-4-fluorophenyl)-4, 5-dihydro-5-(trifluoromethyl-3-isoxazolyl]-N-[2-[(2,2-difluoroethyl)amino]-2-oxoethyl]-5,6-dihydro-); or (b) 3-[(5S)-5-(3,5-dichloro-4-fluorophenyl)-5-(trifluoromethyl)-4,5-dihydroisoxazol-3-yl]-N-[2-[(2, 2-difluoroethyl)amino]-2-oxoethyl]-5, 6-dihydro-4H-cyclopenta[c]thiophene-1-carboxamide).

More particularly, isoxazolines with the following structures are suitable for the methods and formulations of this disclosure:

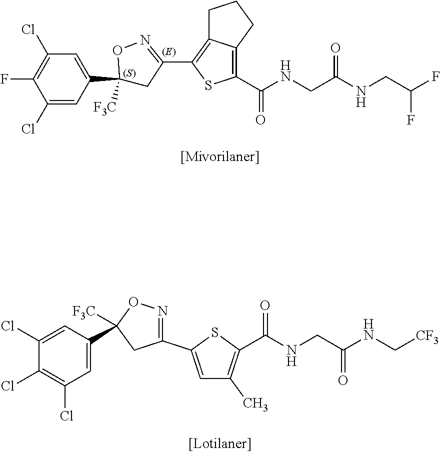

Isoxazolines can react to form salts that are also useful in the methods and formulations of this disclosure. The salts may be prepared using standard procedures for salt preparation. For example, suitable salts can be acid addition salts such as hydrohalogenated acids, e.g., hydrofluoric acid, hydrochloric acid, hydrobromic acid and hydroiodide, nitric acid, sulfuric acid, phosphoric acid, chloric acid, perchloric acid, salts of sulfonic acids, e.g., methanesulfonic acid, ethanesulfonic acid, trifluoromethanesulfonic acid, benzenesulfonic acid, p-toluenesulfonic acid, salts of carboxylic acids, e.g., valeric acids, formic acid, acetic acid, propionic acid, trifluoroacetic acid, fumaric acid, tartaric acid, oxalic acid, maleic acid, malic acid, succinic acid, benzoic acid, mandelic acid, ascorbic acid, lactic acid, gluconic acid, citric acid or salts of amino acids, e.g., glutamic acid and aspartic acid. Alternatively, metal salts are also suitable for the present disclosure. For example, alkali metal salts, e.g., lithium, sodium and potassium, and alkaline earth metals, e.g., calcium, barium and magnesium, or salts of aluminum.

The terms “isoxazoline” and “isoxazoline or a derivative thereof” as used herein refer to any isoxazoline, isoxazoline derivative, a salt thereof, a metabolite thereof, or a combination thereof.

Isoxazolines also provide advantages because they are very effective against ticks with post-treatment residual protection when orally administered in smaller, more frequent/chronic doses. Furthermore, isoxazolines have no known acaricidal cross-resistance to existing compounds. Thus, they are especially useful against tick populations on felines that have existing levels of resistance to currently used products. Isoxazolines, therefore, can be used in integrated pest management (IPM) programs to extend the lifeline of commonly used products where resistance is not well developed or has not yet developed.

Systemic efficacy (e.g., ingestion of blood containing isoxazolines by ticks) provides a different mode of exposure compared to topically applied formulations where contact with the tick at the skin surface is the mode of exposure. The advantages of oral systemic treatments and killing of ticks from their ingestion of blood, compared to topical applications and contact killing, include:
a) reduced exposure to the human applicator and children and objects in the feline's environment (e.g., flooring, carpets, furniture);
b) no worry about loss from exposure of the feline to water (lakes, streams, bathing, etc.) or from loss due to rubbing;
c) no concern about UV exposure and degradation;
d) no problems with oxidation from oils on skin, etc.; and
e) assurance that the entire dose is administered (compared to a topical application where some of the dose may drip off, rub off and/or remain in the dispensing tube immediately after treatment).

The formulations, or feeds, and methods of this disclosure may further include, in combination with the isoxazoline, one or more other active substances having therapeutic efficacy. Such active substances include agents efficacious against ticks. Active substances may include, for example, spinosyns, certain macrocyclic lactones, tick specific chitin synthesis inhibitors, pyridines and pyrazoles or fiproles.

The methods of this disclosure are carried out by administering the isoxazoline to the feline in small, frequent doses. To facilitate routine dosing, the isoxazoline administration may be carried out using a feed or chew. A number of different feeds are envisioned, provided the manufacturing process(es) and feed compositions do not have deleterious effects related to efficacy and safety on the isoxazoline and, if applicable, other active substances. For example, feeds and snacks, chews, treats or supplemental feeds in the broad categories of dry, semi-moist, canned-retorted feeds or fresh refrigerated feeds may be adapted for use with this disclosure. The feline receives a maintenance quantity of isoxazoline by consuming the feed product on a weekly, semi-weekly or daily basis.

By incorporating smaller doses of isoxazoline into an animal feed composition and administering it at an effective rate (most preferably daily), the blood level of isoxazoline rises over time until it reaches an optimal steady state where it can be maintained by a daily or substantially daily dosage. By contrast, when isoxazoline is orally administered in larger doses at lower frequency, e.g., a single treatment of a large dose that is administered via “treat” once in a 30-day period, the level of isoxazoline in the blood spikes shortly after dose administration and then declines until the next dose is administered. The administration of a large dose at low frequency means that the feline must consume more isoxazoline in each dose so that the blood level of isoxazoline does not fall below the necessary level for effective protection before the next dose.

Embodiment 1: A method of controlling a tick infestation in a feline in need thereof, comprising orally administering to said feline an effective amount of an isoxazoline for an effective time at a frequency of at least four times per month.

Embodiment 2: The method of embodiment 1, wherein said feline is a domestic cat.

Embodiment 3: The method of any of embodiment 1 or 2, wherein said isoxazoline is mivorilaner, or a salt thereof.

Embodiment 4: The method of embodiment 3, wherein said mivorilaner is provided in a feed in an amount selected from the group consisting of between about 0.0015 to about 0.6 percent by weight of the feed and between about 0.003 to about 0.36 percent by weight of the feed.

Embodiment 5: The method of any of embodiments 3-4, wherein said mivorilaner is administered to said feline in an amount selected from the group consisting of between about 0.315 mg/kg and about 5 mg/kg of body weight of said feline and between about 0.495 mg/kg and about 2.25 mg/kg of body weight of said feline.

Embodiment 6: The method of any of embodiments 3-5, wherein said administration provides a concentration of mivorilaner of more than about 600 ng/mL and less than about 18,000 ng/mL in said feline's blood for a period of time selected from the group consisting of at least 30 days and at least 365 days.

Embodiment 7: The method of any of embodiment 1 or 2, wherein said isoxazoline is fluralaner, or a salt thereof.

Embodiment 8: The method of embodiment 7, wherein said fluralaner is provided in a feed in an amount selected from the group consisting of between about 0.0003 to about 0.24 percent by weight of the feed and between about 0.0006 to about 0.15 percent by weight of the feed.

Embodiment 9: The method of any of embodiments 7-8, wherein said fluralaner is administered to said feline in an amount selected from the group consisting of between about 0.063 mg/kg and about 1 mg/kg of body weight of said feline and between about 0.1 mg/kg and about 0.45 mg/kg of body weight of said feline.

Embodiment 10: The method of any of embodiments 7-9, wherein said administration provides a concentration of fluralaner of more than about 60 ng/mL and less than about 4500 ng/mL in said feline's blood for a period of time selected from the group consisting of at least 30 days and at least 365 days.

Embodiment 11: The method of any of embodiment 1 or 2, wherein said isoxazoline is sarolaner, or a salt thereof.

Embodiment 12: The method of embodiment 11, wherein said sarolaner is provided in a feed in an amount selected from the group consisting of between about 0.00003 to about 0.24 percent by weight of the feed and between about 0.00006 to about 0.15 percent by weight of the feed.

Embodiment 13: The method of any of embodiments 11-12, wherein said sarolaner is administered to said feline in an amount selected from the group consisting of between about 0.0075 mg/kg and about 0.12 mg/kg of body weight of said feline and between about 0.012 mg/kg and about 0.054 mg/kg of body weight of said feline.

Embodiment 14: The method of any of embodiments 11-13, wherein said administration provides a concentration of sarolaner of more than about 15 ng/mL and less than about 1200 ng/mL in said feline's blood for a period of time selected from the group consisting of at least 30 days and at least 365 days.

Embodiment 15: The method of any of embodiment 1 or 2, wherein said isoxazoline is afoxolaner, or a salt thereof.

Embodiment 16: The method of embodiment 15, wherein said afoxolaner is provided in a feed in an amount selected from the group consisting of between about 0.000075 to about 0.24 percent by weight of the feed and between about 0.00015 to about 0.15 percent by weight of the feed.

Embodiment 17: The method of any of embodiments 15-16, wherein said afoxolaner is administered to said feline in an amount selected from the group consisting of between about 0.015 mg/kg and about 0.25 mg/kg of body weight of said feline and between about 0.0255 mg/kg and about 0.113 mg/kg of body weight of said feline.

Embodiment 18: The method of any of embodiments 15-17, wherein said administration provides a concentration of afoxolaner of more than about 30 ng/mL and less than about 1800 ng/mL in said feline's blood for a period of time selected from the group consisting of at least 30 days and at least 365 days.

Embodiment 19: The method of any of embodiment 1 or 2, wherein said isoxazoline is lotilaner, or a salt thereof.

Embodiment 20: The method of embodiment 19, wherein said lotilaner is provided in a feed in an amount selected from the group consisting of between about 0.0006 to about 0.24 percent by weight of the feed and between about 0.0012 to about 0.15 percent by weight of the feed.

Embodiment 21: The method of any of embodiments 19-20, wherein said lotilaner is administered to said feline in an amount selected from the group consisting of between about 0.124 mg/kg and about 2 mg/kg of body weight of said feline and between about 0.2 mg/kg and about 0.9 mg/kg of body weight of said feline.

Embodiment 22: The method of any of embodiments 19-21, wherein said administration provides a concentration of lotilaner of more than about 120 ng/mL and less than about 4500 ng/mL in said feline's blood for a period of time selected from the group consisting of at least 30 days and at least 365 days.

Embodiment 23: The method of any of embodiments 1-22, wherein said isoxazoline is administered as a component of a feed.

Embodiment 24: The method of embodiment 23, wherein said feed is dry cat food.

Embodiment 25: The method of embodiment 23, wherein said feed is wet cat food.

Embodiment 26: The method of any of embodiments 1-22, wherein said isoxazoline is administered as a component of a chew.

Embodiment 27: The method of any of embodiments 1-26, wherein said frequency is selected from the group consisting of at least 3 times per week, substantially daily and daily.

Embodiment 28: The method of any of embodiments 1-27, wherein said effective time comprises administering the isoxazoline for a time period selected from the group consisting of at least one week and at least two weeks.

Embodiment 29: The method of any of embodiments 1-28, wherein said administration provides a therapeutically effective level of isoxazoline in said feline's blood within a time period selected from the group consisting of one week of the first administration of said isoxazoline and two days of the first administration of said isoxazoline.

Embodiment 30: The method of any of embodiments 1-29, wherein said administration provides a therapeutically effective level of isoxazoline in said feline's blood for a time period selected from the group consisting of at least 30 days, at least 60 days, at least 90 days, at least 180 days and at least 365 days.

Embodiment 31: The method of any of embodiments 1-30, wherein said isoxazoline is administered for a period of time selected from the group consisting of at least 15 out of 30 days and at least 20 out of 30 days.

Embodiment 32: The method of any of embodiments 1-31, wherein said isoxazoline is administered as a component of a feed that comprises one or more other active substances.

Embodiment 33: The method of any of embodiments 1-32, further comprising discontinuing the administration of the isoxazoline for a period of time selected from the group consisting of at least 3 days and at least 7 days, wherein the feline's blood concentration of isoxazoline is maintained at a therapeutically effective level.

Embodiment 34: The method of embodiment 33, further comprising resuming the administration of the isoxazoline after the discontinuing of the administration of the isoxazoline and thereby maintaining the feline's blood concentration of isoxazoline at the therapeutically effective level.

Embodiment 35: An isoxazoline for use in controlling ticks on a feline in need thereof, said isoxazoline being administered in an effective amount to said feline for an effective time at a frequency of at least four times per month.

Embodiment 36: The isoxazoline of embodiment 35, wherein said feline is a domestic cat.

Embodiment 37: The isoxazoline of any of embodiment 35 or 36, wherein said isoxazoline is mivorilaner, or a salt thereof.

Embodiment 38: The isoxazoline of embodiment 37, wherein said mivorilaner is provided in a feed in an amount selected from the group consisting of between about 0.0015 to about 0.6 percent by weight of the feed and about 0.003 to about 0.36 percent by weight of the feed.

Embodiment 39: The isoxazoline of any of embodiments 37-38, wherein said mivorilaner is administered to said feline in an amount selected from the group consisting of between about 0.315 mg/kg and about 5 mg/kg of body weight of said feline and between about 0.495 mg/kg and about 2.25 mg/kg of body weight of said feline.

Embodiment 40: The isoxazoline of any of embodiments 37-39, wherein said administration provides a concentration of mivorilaner of more than about 600 ng/mL and less than about 18,000 ng/mL in said feline's blood for a period of time selected from the group consisting of at least 30 days and at least 365 days.

Embodiment 41: The isoxazoline of any of embodiment 35 or 36, wherein said isoxazoline is fluralaner, or a salt thereof.

Embodiment 42: The isoxazoline of embodiment 41, wherein said fluralaner is provided in a feed in an amount selected from the group consisting of between about 0.0003 to about 0.24 percent by weight of the feed and between about 0.0006 to about 0.15 percent by weight of the feed.

Embodiment 43: The isoxazoline of any of embodiments 41-42, wherein said fluralaner is administered to said feline in an amount selected from the group consisting of between about 0.063 mg/kg and about 1 mg/kg of body weight of said feline and between about 0.1 mg/kg and about 0.45 mg/kg of body weight of said feline.

Embodiment 44: The isoxazoline of any of embodiments 41-43, wherein said administration provides a concentration of fluralaner of more than about 60 ng/mL and less than about 4500 ng/mL in said feline's blood for a period of time selected from the group consisting of at least 30 days and at least 365 days.

Embodiment 45: The isoxazoline of any of embodiment 35 or 36, wherein said isoxazoline is sarolaner, or a salt thereof.

Embodiment 46: The isoxazoline of embodiment 45, wherein said sarolaner is provided in a feed in an amount selected from the group consisting of between about 0.00003 to about 0.24 percent by weight of the feed and between about 0.00006 to about 0.15 percent by weight of the feed.

Embodiment 47: The isoxazoline of any of embodiments 45-46, wherein said sarolaner is administered to said feline in an amount selected from the group consisting of between about 0.0075 mg/kg and about 0.12 mg/kg of body weight of said feline and between about 0.012 mg/kg and about 0.054 mg/kg of body weight of said feline.

Embodiment 48: The isoxazoline of any of embodiments 45-47, wherein said administration provides a concentration of sarolaner of more than about 15 ng/mL and less than about 1200 ng/mL in said feline's blood for a period of time selected from the group consisting of at least 30 days and at least 365 days.

Embodiment 49: The isoxazoline of any of embodiment 35 or 36, wherein said isoxazoline is afoxolaner, or a salt thereof.

Embodiment 50: The isoxazoline of embodiment 49, wherein said afoxolaner is provided in a feed in an amount selected from the group consisting of between about 0.000075 to about 0.24 percent by weight of the feed and between about 0.00015 to about 0.15 percent by weight of the feed.

Embodiment 51: The isoxazoline of any of embodiments 49-50, wherein said afoxolaner is administered to said feline in an amount selected from the group consisting of between about 0.015 mg/kg and about 0.25 mg/kg of body weight of said feline and between about 0.0255 mg/kg and about 0.113 mg/kg of body weight of said feline.

Embodiment 52: The isoxazoline of any of embodiments 49-51, wherein said administration provides a concentration of afoxolaner of more than about 30 ng/mL and less than about 1800 ng/mL in said feline's blood for a period of time selected from the group consisting of at least 30 days and at least 365 days.

Embodiment 53: The isoxazoline of any of embodiment 35 or 36, wherein said isoxazoline is lotilaner, or a salt thereof.

Embodiment 54: The isoxazoline of embodiment 53, wherein said lotilaner is provided in a feed in an amount selected from the group consisting of between about 0.0006 to about 0.24 percent by weight of the feed and between about 0.0012 to about 0.15 percent by weight of the feed.

Embodiment 55: The isoxazoline of any of embodiments 53-54, wherein said lotilaner is administered to said feline in an amount selected from the group consisting of between about 0.124 mg/kg and about 2 mg/kg of body weight of said feline and between about 0.2 mg/kg and about 0.9 mg/kg of body weight of said feline.

Embodiment 56: The isoxazoline of any of embodiments 53-55, wherein said administration provides a concentration of lotilaner of more than about 120 ng/mL and less than about 4500 ng/mL in said feline's blood for a period of time selected from the group consisting of at least 30 days and at least 365 days.

Embodiment 57: The isoxazoline of any of embodiments 35-56, wherein said isoxazoline is administered as a component of a feed.

Embodiment 58: The isoxazoline of embodiment 57, wherein said feed is dry cat food.

Embodiment 59: The isoxazoline of any of embodiment 57, wherein said feed is wet cat food.

Embodiment 60: The isoxazoline of any of embodiments 35-56, wherein said isoxazoline is administered as a component of a chew.

Embodiment 61: The isoxazoline of any of embodiments 35-60, wherein said feeding frequency is selected from the group consisting of at least 3 times per week, substantially daily and daily.

Embodiment 62: The isoxazoline of any of embodiments 35-61, wherein said effective time comprises administering the isoxazoline for a period of time selected from the group consisting of at least one week and at least two weeks.

Embodiment 63: The isoxazoline of any of embodiments 35-62, wherein said administration provides a therapeutically effective level of isoxazoline in said feline's blood within a period of time selected from the group consisting of one week of the first administration of said isoxazoline and two days of the first administration of said isoxazoline.

Embodiment 64: The isoxazoline of any of embodiments 35-63, wherein said administration provides a therapeutically effective level of isoxazoline in said feline's blood for a period of time selected from the group consisting of at least 30 days, at least 60 days, at least 90 days, at least 180 days and at least 365 days.

Embodiment 65: The isoxazoline of any of embodiments 35-64, wherein said isoxazoline is administered at a frequency selected from the group consisting of at least 15 out of 30 days and at least 20 out of 30 days.

Embodiment 66: The isoxazoline of any of embodiments 35-65, wherein said isoxazoline is administered as a component of a feed that comprises one or more other active substances.

Embodiment 67: The isoxazoline of any of embodiments 35-66, further comprising discontinuing the administration of the isoxazoline for a period of time selected from the group consisting of at least 3 days and at least 7 days, wherein the feline's blood concentration of isoxazoline is maintained at a therapeutically effective level.

Embodiment 68: The isoxazoline of embodiment 67, further comprising resuming the administration of the isoxazoline after the discontinuing of the administration of the isoxazoline and thereby maintaining the feline's blood concentration of isoxazoline at the therapeutically effective level.

Embodiment 69: A feed or chew for controlling ticks in a feline, said feed or chew comprising an effective amount of an isoxazoline to control a tick infestation when administered to said feline for an effective time at a frequency of at least four times per month.

Embodiment 70: The feed or chew of embodiment 69, wherein said feline is a domestic cat.

Embodiment 71: The feed or chew of any of embodiment 69 or 70, wherein said isoxazoline is mivorilaner, or a salt thereof.

Embodiment 72: The feed or chew of embodiment 71, wherein said mivorilaner is provided in a feed in an amount selected from the group consisting of between about 0.0015 to about 0.6 percent by weight of the feed and between about 0.003 to about 0.36 percent by weight of the feed.

Embodiment 73: The feed or chew of any of embodiments 71-72, wherein said mivorilaner is administered to said feline in an amount selected from the group consisting of between about 0.315 mg/kg and about 5 mg/kg of body weight of said feline and between about 0.495 mg/kg and about 2.25 mg/kg of body weight of said feline.

Embodiment 74: The feed or chew of any of embodiments 71-73, wherein said administration provides a concentration of mivorilaner of more than about 600 ng/mL and less than about 18,000 ng/mL in said feline's blood for a period of time selected from the group consisting of at least 30 days and at least 365 days.

Embodiment 75: The feed or chew of any of embodiment 69 or 70, wherein said isoxazoline is fluralaner, or a salt thereof.

Embodiment 76: The feed or chew of embodiment 75, wherein said fluralaner is provided in a feed in an amount selected from the group consisting of between about 0.0003 to about 0.24 percent by weight of the feed and between about 0.0006 to about 0.15 percent by weight of the feed.

Embodiment 77: The feed or chew of any of embodiments 75 or 76, wherein said fluralaner is administered to said feline in an amount selected from the group consisting of between about 0.063 mg/kg and about 1 mg/kg of body weight of said feline and between about 0.1 mg/kg and about 0.45 mg/kg of body weight of said feline.

Embodiment 78: The feed or chew of any of embodiments 75-77, wherein said administration provides a concentration of fluralaner of more than about 60 ng/mL and less than about 4500 ng/mL in said feline's blood for a period of time selected from the group consisting of at least 30 days and at least 365 days.

Embodiment 79: The feed or chew of any of embodiment 69 or 70, wherein said isoxazoline is sarolaner, or a salt thereof.

Embodiment 80: The feed or chew of embodiment 79, wherein said sarolaner is provided in a feed in an amount selected from the group consisting of between about 0.00003 to about 0.24 percent by weight of the feed and between about 0.00006 to about 0.15 percent by weight of the feed.

Embodiment 81: The feed or chew of any of embodiments 79 or 80, wherein said sarolaner administered to said feline in an amount selected from the group consisting of between about 0.0075 mg/kg and about 0.12 mg/kg of body weight of said feline and between about 0.012 mg/kg and about 0.054 mg/kg of body weight of said feline.

Embodiment 82: The feed or chew of any of embodiments 79-81, wherein said administration provides a concentration of sarolaner of more than about 15 ng/mL and less than about 1200 ng/mL in said feline's blood for a period of time selected from the group consisting of at least 30 days and at least 365 days.

Embodiment 83: The feed or chew of any of embodiment 69 or 70, wherein said isoxazoline is afoxolaner, or a salt thereof.

Embodiment 84: The feed or chew of embodiment 83, wherein said afoxolaner is provided in a feed in an amount selected from the group consisting of between about 0.000075 to about 0.24 percent by weight of the feed and between about 0.00015 to about 0.15 percent by weight of the feed.

Embodiment 85: The feed or chew of any of embodiments 83 or 84, wherein said afoxolaner is administered to said feline in an amount selected from the group consisting of between about 0.015 mg/kg and about 0.25 mg/kg of body weight of said feline and between about 0.0255 mg/kg and about 0.113 mg/kg of body weight of said feline.

Embodiment 86: The feed or chew of any of embodiments 83-85, wherein said administration provides a concentration of afoxolaner of more than about 30 ng/mL and less than about 1800 ng/mL in said feline's blood for a period of time selected from the group consisting of at least 30 days and at least 365 days.

Embodiment 87: The feed or chew of any of embodiment 69 or 70, wherein said isoxazoline is lotilaner, or a salt thereof.

Embodiment 88: The feed or chew of embodiment 87, wherein said lotilaner is provided in a feed in an amount selected from the group consisting of between about 0.0006 to about 0.24 percent by weight of the feed and between about 0.0012 to about 0.15 percent by weight of the feed.

Embodiment 89: The feed or chew of any of embodiments 87 or 88, wherein said lotilaner is administered to said feline in an amount selected from the group consisting of between about 0.124 mg/kg and about 2 mg/kg of body weight of said feline and between about 0.2 mg/kg and about 0.9 mg/kg of body weight of said feline.

Embodiment 90: The feed or chew of any of embodiments 87-89, wherein said administration provides a concentration of lotilaner of more than about 120 ng/mL and less than about 4500 ng/mL in said feline's blood for a period of time selected from the group consisting of at least 30 days and at least 365 days.

Embodiment 91: The feed or chew of any of embodiments 69-90, wherein said feed is dry cat food.

Embodiment 92: The feed or chew of any of embodiments 69-90, wherein said feed is wet cat food.

Embodiment 93: The feed or chew of any of embodiments 69-92, wherein said frequency is selected from the group consisting of at least 3 times per week, substantially daily and daily.

Embodiment 94: The feed or chew of any of embodiments 69-93, wherein said effective time comprises administering the feed or chew for a period of time selected from the group consisting of at least one week and at least two weeks.

Embodiment 95: The feed or chew of any of embodiments 69-94, wherein said administration provides a therapeutically effective level of isoxazoline in said feline's blood within a period of time selected from the group consisting of one week of the first administration of said feed or chew and two days of the first administration of said feed or chew.

Embodiment 96: The feed or chew of any of embodiments 69-95, wherein said administration provides a therapeutically effective level of isoxazoline in said feline's blood for a period of time selected from the group consisting of at least 30 days, at least 60 days, at least 90 days, at least 180 days and at least 365 days.

Embodiment 97: The feed or chew of any of embodiments 69-96, wherein said feed or chew is administered at a frequency selected from the group consisting of at least 15 out of 30 days and at least 20 out of 30 days.

Embodiment 98: The feed or chew of any of embodiments 69-97, wherein said feed or chew comprises one or more other active substances.

Embodiment 99: The feed or chew of any of embodiments 69-98, further comprising discontinuing the administration of the feed or chew for a period of time selected from the group consisting of at least 3 days and at least 7 days, wherein the feline's blood concentration of isoxazoline is maintained at a therapeutically effective level.

Embodiment 100: The feed or chew of embodiment 99, further comprising resuming the administration of the feed or chew after the discontinuing of the administration of the feed or chew and thereby maintaining the feline's blood concentration of isoxazoline at the therapeutically effective level.

Embodiment 101: The method of any of embodiment 1 or 2, wherein said isoxazoline is umifoxolaner, or a salt thereof.

Embodiment 102: The method of embodiment 101, wherein said umifoxolaner is provided in a feed in an amount selected from the group consisting of between about 0.00003 to about 0.24 percent by weight of the feed and between about 0.000075 to about 0.15 percent by weight of the feed.

Embodiment 103: The method of any of embodiments 101-102, wherein said umifoxolaner is administered to said feline in an amount selected from the group consisting of between about 0.0075 mg/kg and about 0.12 mg/kg of body weight of said feline and between about 0.012 mg/kg and about 0.056 mg/kg of body weight of said feline.

Embodiment 104: The method of any of embodiments 101-103, wherein said administration provides a concentration of umifoxolaner of more than about 15 ng/mL and less than about 1200 ng/mL in said feline's blood for a period of time selected from the group consisting of at least 30 days and at least 365 days.

Embodiment 105: The method of any of embodiment 1 or 2, wherein said isoxazoline is esafoxolaner, or a salt thereof.

Embodiment 106: The method of embodiment 105, wherein said esafoxolaner is provided in a feed in an amount selected from the group consisting of between about 0.00003 to about 0.24 percent by weight of the feed and between about 0.000075 to about 0.15 percent by weight of the feed.

Embodiment 107: The method of any of embodiments 105-106, wherein said esafoxolaner is administered to said feline in an amount selected from the group consisting of between about 0.0075 mg/kg and about 0.12 mg/kg of body weight of said feline and between about 0.012 mg/kg and about 0.0563 mg/kg of body weight of said feline.

Embodiment 108: The method of any of embodiments 105-107, wherein said administration provides a concentration of esafoxolaner of more than about 15 ng/mL and less than about 900 ng/mL in said feline's blood for a period of time selected from the group consisting of at least 30 days and at least 365 days.

Embodiment 109: The method of any of embodiment 1 or 2, wherein said isoxazoline is tigolaner, or a salt thereof.

Embodiment 110: The method of embodiment 109, wherein said tigolaner is provided in a feed in an amount selected from the group consisting of between about 0.00003 to about 0.24 percent by weight of the feed and between about 0.000075 to about 0.15 percent by weight of the feed.

Embodiment 111: The method of any of embodiments 109-110, wherein said tigolaner is administered to said feline in an amount selected from the group consisting of between about 0.0075 mg/kg and about 0.12 mg/kg of body weight of said feline and between about 0.012 mg/kg and about 0.0563 mg/kg of body weight of said feline.

Embodiment 112: The method of any of embodiments 109-111, wherein said administration provides a concentration of tigolaner of more than about 15 ng/mL and less than about 900 ng/mL in said feline's blood for a period of time selected from the group consisting of at least 30 days and at least 365 days.

Embodiment 113: The isoxazoline of any of embodiment 35 or 36, wherein said isoxazoline is umifoxolaner, or a salt thereof.

Embodiment 114: The isoxazoline of embodiment 113, wherein said umifoxolaner is provided in a feed in an amount selected from the group consisting of between about 0.00003 to about 0.24 percent by weight of the feed and between about 0.000075 to about 0.15 percent by weight of the feed.

Embodiment 115: The isoxazoline of any of embodiments 113-114, wherein said umifoxolaner is administered to said feline in an amount selected from the group consisting of between about 0.0075 mg/kg and about 0.12 mg/kg of body weight of said feline and between about 0.012 mg/kg and about 0.056 mg/kg of body weight of said feline.

Embodiment 116: The isoxazoline of any of embodiments 113-115, wherein said administration provides a concentration of umifoxolaner of more than about 15 ng/mL and less than about 1200 ng/mL in said feline's blood for a period of time selected from the group consisting of at least 30 days and at least 365 days.

Embodiment 117: The isoxazoline of any of embodiment 35 or 36, wherein said isoxazoline is esafoxolaner, or a salt thereof.

Embodiment 118: The isoxazoline of embodiment 117, wherein said esafoxolaner is provided in a feed in an amount selected from the group consisting of between about 0.00003 to about 0.24 percent by weight of the feed and between about 0.000075 to about 0.15 percent by weight of the feed.

Embodiment 119: The isoxazoline of any of embodiments 117-118, wherein said esafoxolaner is administered to said feline in an amount selected from the group consisting of between about 0.0075 mg/kg and about 0.12 mg/kg of body weight of said feline and between about 0.012 mg/kg and about 0.0563 mg/kg of body weight of said feline.

Embodiment 120: The isoxazoline of any of embodiments 118-119, wherein said administration provides a concentration of esafoxolaner of more than about 15 ng/mL and less than about 900 ng/mL in said feline's blood for a period of time selected from the group consisting of at least 30 days and at least 365 days.

Embodiment 121: The isoxazoline of any of embodiment 35 or 36, wherein said isoxazoline is tigolaner, or a salt thereof.

Embodiment 122: The isoxazoline of embodiment 121, wherein said tigolaner is provided in a feed in an amount selected from the group consisting of between about 0.00003 to about 0.24 percent by weight of the feed and between about 0.000075 to about 0.15 percent by weight of the feed.

Embodiment 123: The isoxazoline of any of embodiments 121-122, wherein said tigolaner is administered to said feline in an amount selected from the group consisting of between about 0.0075 mg/kg and about 0.12 mg/kg of body weight of said feline and between about 0.012 mg/kg and about 0.0563 mg/kg of body weight of said feline.

Embodiment 124: The isoxazoline of any of embodiments 121-123, wherein said administration provides a concentration of tigolaner of more than about 15 ng/mL and less than about 900 ng/mL in said feline's blood for a period of time selected from the group consisting of at least 30 days and at least 365 days.

Embodiment 125: The feed or chew of any of embodiment 69 or 70, wherein said isoxazoline is umifoxolaner, or a salt thereof.

Embodiment 126: The feed or chew of embodiment 125, wherein said umifoxolaner is provided in a feed in an amount selected from the group consisting of between about 0.00003 to about 0.24 percent by weight of the feed and between about 0.000075 to about 0.15 percent by weight of the feed.

Embodiment 127: The feed or chew of any of embodiments 125-126, wherein said umifoxolaner is administered to said feline in an amount selected from the group consisting of between about 0.0075 mg/kg and about 0.12 mg/kg of body weight of said feline and between about 0.012 mg/kg and about 0.056 mg/kg of body weight of said feline.

Embodiment 128: The feed or chew of any of embodiments 125-127, wherein said administration provides a concentration of umifoxolaner of more than about 15 ng/mL and less than about 1200 ng/mL in said feline's blood for a period of time selected from the group consisting of at least 30 days and at least 365 days.

Embodiment 129: The feed or chew of any of embodiment 69 or 70, wherein said isoxazoline is esafoxolaner, or a salt thereof.

Embodiment 130: The feed or chew of embodiment 129, wherein said esafoxolaner is provided in a feed in an amount selected from the group consisting of between about 0.00003 to about 0.24 percent by weight of the feed and between about 0.00005 to about 0.1 percent by weight of the feed.

Embodiment 131: The feed or chew of any of embodiments 129-130, wherein said esafoxolaner is administered to said feline in an amount selected from the group consisting of between about 0.0075 mg/kg and about 0.12 mg/kg of body weight of said feline and between about 0.012 mg/kg and about 0.0563 mg/kg of body weight of said feline.

Embodiment 132: The feed or chew of any of embodiments 129-131, wherein said administration provides a concentration of esafoxolaner of more than about 15 ng/mL and less than about 900 ng/mL in said feline's blood for a period of time selected from the group consisting of at least 30 days and at least 365 days.

Embodiment 133: The feed or chew of any of embodiment 69 or 70, wherein said isoxazoline is tigolaner, or a salt thereof.

Embodiment 134: The feed or chew of embodiment 133, wherein said tigolaner is provided in a feed in an amount selected from the group consisting of between about 0.00003 to about 0.24 percent by weight of the feed and between about 0.000075 to about 0.15 percent by weight of the feed.

Embodiment 135: The feed or chew of any of embodiments 133-134, wherein said tigolaner is administered to said feline in an amount selected from the group consisting of between about 0.0075 mg/kg and about 0.12 mg/kg of body weight of said feline and between about 0.012 mg/kg and about 0.0563 mg/kg of body weight of said feline.

Embodiment 136: The feed or chew of any of embodiments 133-135, wherein said administration provides a concentration of tigolaner of more than about 15 ng/mL and less than about 900 ng/mL in said feline's blood for a period of time selected from the group consisting of at least 30 days and at least 365 days.

Embodiment 137: The method of any of embodiment 1 or 2, wherein said isoxazoline is modolaner, or a salt thereof.

Embodiment 138: The method of embodiment 137, wherein said modolaner is provided in a feed in an amount selected from the group consisting of between about 0.000075 to about 0.24 percent by weight of the feed and between about 0.00015 to about 0.15 percent by weight of the feed.

Embodiment 139: The method of any of embodiments 137-138, wherein said modolaner is administered to said feline in an amount selected from the group consisting of between about 0.015 mg/kg and about 0.25 mg/kg of body weight of said feline and between about 0.0255 mg/kg and about 0.113 mg/kg of body weight of said feline.

Embodiment 140: The method of any of embodiments 137-139, wherein said administration provides a concentration of modolaner of more than about 30 ng/mL and less than about 1800 ng/mL in said feline's blood for a period of time selected from the group consisting of at least 30 days and at least 365 days.

Embodiment 141: The isoxazoline of any of embodiment 35 or 36, wherein said isoxazoline is modolaner, or a salt thereof.

Embodiment 142: The isoxazoline of embodiment 141, wherein said modolaner is provided in a feed in an amount selected from the group consisting of between about 0.000075 to about 0.24 percent by weight of the feed and between about 0.00015 to about 0.15 percent by weight of the feed.

Embodiment 143: The isoxazoline of any of embodiments 141-142, wherein said modolaner is administered to said feline in an amount selected from the group consisting of between about 0.015 mg/kg and about 0.25 mg/kg of body weight of said feline and between about 0.0255 mg/kg and about 0.113 mg/kg of body weight of said feline.

Embodiment 144: The isoxazoline of any of embodiments 141-143, wherein said administration provides a concentration of modolaner of more than about 30 ng/mL and less than about 1800 ng/mL in said feline's blood for a period of time selected from the group consisting of at least 30 days and at least 365 days.

Embodiment 145: The feed or chew of any of embodiment 69 or 70, wherein said isoxazoline is modolaner, or a salt thereof.

Embodiment 146: The feed or chew of embodiment 145, wherein said modolaner is provided in a feed in an amount selected from the group consisting of between about 0.000075 to about 0.24 percent by weight of the feed and between about 0.00015 to about 0.15 percent by weight of the feed.

Embodiment 147: The feed or chew of any of embodiments 145-146, wherein said modolaner is administered to said feline in an amount selected from the group consisting of between about 0.015 mg/kg and about 0.25 mg/kg of body weight of said feline and between about 0.0255 mg/kg and about 0.113 mg/kg of body weight of said feline.

Embodiment 148: The feed or chew of any of embodiments 145-147, wherein said administration provides a concentration of modolaner of more than about 30 ng/mL and less than about 1800 ng/mL in said feline's blood for a period of time selected from the group consisting of at least 30 days and at least 365 days.

In an aspect of any of the above embodiments, administration provides a therapeutically effective concentration of the particular isoxazoline in said feline's blood for at least 30 days. The precise concentration may vary according to the particular isoxazoline. For example, administration of mivorilaner provides a concentration of isoxazoline of more than about 600 ng/mL and less than about 18,000 ng/mL in said feline's blood for at least 30 days. More preferably, administration of mivorilaner provides a concentration of isoxazoline of more than about 600 ng/mL and less than about 12,000 ng/mL in said feline's blood for at least 30 days. In another example, afoxolaner provides a concentration of isoxazoline of more than about 30 ng/mL and less than about 1800 ng/mL in said feline's blood for at least 30 days. More preferably, afoxolaner provides a concentration of isoxazoline of more than about 30 ng/mL and less than about 1200 ng/mL in said feline's blood for at least 30 days. In another example, fluralaner provides a concentration of isoxazoline of more than about 60 ng/mL and less than about 4500 ng/mL in said feline's blood for at least 30 days. More preferably, fluralaner provides a concentration of isoxazoline of more than about 60 ng/mL and less than about 3000 ng/mL in said feline's blood for at least 30 days. In another example, sarolaner provides a concentration of isoxazoline of more than about 15 ng/mL and less than about 1200 ng/mL in said feline's blood for at least 30 days. More preferably, sarolaner provides a concentration of isoxazoline of more than about 15 ng/mL and less than about 900 ng/mL in said feline's blood for at least 30 days. In another example, lotilaner provides a concentration of isoxazoline of more than about 120 ng/mL and less than about 4500 ng/mL in said feline's blood for at least 30 days. More preferably, lotilaner provides a concentration of isoxazoline of more than about 120 ng/mL and less than about 3000 ng/mL in said feline's blood for at least 30 days. In another example, tigolaner provides a concentration of isoxazoline of more than about 15 ng/mL and less than about 1200 ng/mL in said feline's blood for at least 30 days. More preferably, tigolaner provides a concentration of isoxazoline of more than about 15 ng/mL and less than about 900 ng/mL in said feline's blood for at least 30 days. In another example, umifoxolaner provides a concentration of isoxazoline of more than about 15 ng/mL and less than about 1200 ng/mL in said feline's blood for at least 30 days. More preferably, umifoxolaner provides a concentration of isoxazoline of more than about 15 ng/mL and less than about 900 ng/mL in said feline's blood for at least 30 days. In another example, esafoxolaner provides a concentration of isoxazoline of more than about 15 ng/mL and less than about 900 ng/mL in said feline's blood for at least 30 days. More preferably, esafoxolaner provides a concentration of isoxazoline of more than about 15 ng/mL and less than about 600 ng/mL in said feline's blood for at least 30 days.

In an aspect of any of the above embodiments, administration provides a therapeutically effective concentration of the particular isoxazoline in said feline's blood for at least 365 days. The precise concentration may vary according to the particular isoxazoline. For example, administration of mivorilaner provides a concentration of isoxazoline of more than about 600 ng/mL and less than about 18,000 ng/mL in said feline's blood for at least 365 days. More preferably, administration of mivorilaner provides a concentration of isoxazoline of more than about 600 ng/mL and less than about 12,000 ng/mL in said feline's blood for at least 365 days. In another example, afoxolaner provides a concentration of isoxazoline of more than about 30 ng/mL and less than about 1800 ng/mL in said feline's blood for at least 365 days. More preferably, afoxolaner provides a concentration of isoxazoline of more than about 30 ng/mL and less than about 1200 ng/mL in said feline's blood for at least 365 days. In another example, fluralaner provides a concentration of isoxazoline of more than about 60 ng/mL and less than about 4500 ng/mL in said feline's blood for at least 365 days. More preferably, fluralaner provides a concentration of isoxazoline of more than about 60 ng/mL and less than about 3000 ng/mL in said feline's blood for at least 365 days. In another example, sarolaner provides a concentration of isoxazoline of more than about 15 ng/mL and less than about 1200 ng/mL in said feline's blood for at least 365 days. More preferably, sarolaner provides a concentration of isoxazoline of more than about 15 ng/mL and less than about 900 ng/mL in said feline's blood for at least 365 days. In another example, lotilaner provides a concentration of isoxazoline of more than about 120 ng/mL and less than about 4500 ng/mL in said feline's blood for at least 365 days. More preferably, lotilaner provides a concentration of isoxazoline of more than about 120 ng/mL and less than about 3000 ng/mL in said feline's blood for at least 365 days. In another example, tigolaner provides a concentration of isoxazoline of more than about 15 ng/mL and less than about 1200 ng/mL in said feline's blood for at least 365 days. More preferably, tigolaner provides a concentration of isoxazoline of more than about 15 ng/mL and less than about 900 ng/mL in said feline's blood for at least 365 days. In another example, umifoxolaner provides a concentration of isoxazoline of more than about 15 ng/mL and less than about 1200 ng/mL in said feline's blood for at least 365 days. More preferably, umifoxolaner provides a concentration of isoxazoline of more than about 15 ng/mL and less than about 900 ng/mL in said feline's blood for at least 365 days. In another example, esafoxolaner provides a concentration of isoxazoline of more than about 15 ng/mL and less than about 900 ng/mL in said feline's blood for at least 365 days. More preferably, esafoxolaner provides a concentration of isoxazoline of more than about 15 ng/mL and less than about 600 ng/mL in said feline's blood for at least 365 days.

### BRIEF DESCRIPTION OF THE FIGURES

FIG. 1. A graph of the arithmetic mean efficacy for treatment Group 2 (stars) and Group 3 (horizontal hash). Percent efficacy=[AM fleas CONTROL−AM fleas TREATED]/AM fleas CONTROL. Efficacy measured at 2, 16, 27, 30, and 37 days after Initial Treatment, with either 0.75 mg of Mivorilaner/kg (Group 2) or 1.5 mg of Mivorilaner/kg (Group 3).

### DESCRIPTION

All ratios, percentages, and parts discussed herein are “by weight” unless otherwise specified.

The term “controlling a tick infestation” refers to preventing, minimizing or eliminating an infestation by ticks on a feline.

The term “tick” refers to any member of the order Ixodida. The term “tick” includes the egg, larval, nymph, and adult stages of development. More particularly, the term tick includes ticks of the families Ixodidae and Argasidae. More particularly, the term “tick” includes species of the genera Africaniella, Amblyomma, Anomalohimalaya, Bothriocroton, Dermacentor, Haemaphysalis, Hyalomma, Ixodes, Margaropus, Nosomma, Rhipicentor, Rhipicephalus, Antricola, Argas, Nothoaspis, Ornithodoros, and Otobius.

The term “feline” refers to any member of the subfamily Felidae, which includes such species as the domestic cat, bobcats, wildcats, ocelots, members of the genus lynx, Pallas's cat and cougars.

In carrying out the methods of this disclosure, a “feed” is an animal feed, snack, treat or other supplemental feed that may be administered daily or substantially daily. By using different forms of feed, e.g., kibble and treats, a pet owner may vary the feline's meals and snacks from time to time while still conveniently administering a daily dose of isoxazoline.

The term “chew” refers to a treat that has flavor and aromatic properties that are appealing to a feline, but typically has no nutritional value. In carrying out the methods of this disclosure, a “feed” and/or a “chew” may be used interchangeably.

The term “effective time,” also referred to herein as “effective duration,” for the purposes of this disclosure includes at least the duration of administration needed to bring the level of isoxazoline in the feline's blood to a sufficiently high level for controlling ticks, i.e., a “therapeutically effective” level. In some embodiments, the effective time may be as little as three days. In other instances, the effective time may be seven days or fifteen days or longer. As discussed below, the effective time will vary based on how frequently the feed or isoxazoline is administered.

As just alluded, the “effective time” will vary as a function of the frequency at which the isoxazoline is administered. The term “effective frequency” as used herein means the number of doses over a given time that produce a therapeutically effective concentration of isoxazoline in the feline's blood. In all events, the term “effective frequency” as used herein contemplates multiple doses of the isoxazoline per month. One of skill in the art will appreciate that the isoxazoline may be administered in a range of frequencies. For example, the isoxazoline may be administered at a frequency of daily, every other day, every third day, once per week or even at inconsistent time intervals.

Further, as discussed above, the effective frequency may affect the duration required to obtain a therapeutically effective level of isoxazoline in the feline's blood. By way of example, if the feline were being fed the feed composition daily, the duration of administration required to achieve a therapeutically effective level of isoxazoline in the feline's blood, and thus the “effective time,” would be comparatively less than if the feline were being fed the feed composition only once or twice per week.

Further, the effective frequency is influenced by the amount of the daily dose in mg/kg of body weight of the feline. Particularly, at slightly higher daily doses, missed doses have less of an impact on efficacy.

Further, the effective frequency is influenced by the duration of treatment. In the initial stages, e.g., before the amount of isoxazoline in the feline's blood has reached a therapeutically effective level, the animal feed may need to be administered more often than would be necessary after a longer period of use, i.e., once a therapeutically effective level is obtained.

For purposes of this disclosure, “substantially daily” means a sufficiently regular basis such that the isoxazoline concentration in the feline's blood rises to and remains at a therapeutically effective level. For example, the disclosed feed composition can preferably be fed to a feline every day indefinitely. However, as a practical matter, there are many reasons why days may be missed or skipped periodically. For example, the feline may be ill or the owner may run out of the medicated feed composition. The disclosed method is robust enough that the feline will still be protected from ticks to some extent even with occasional interruptions in doses of isoxazoline. In carrying out the method of this disclosure, the term “substantially daily” includes at least 10 days per month, more preferably at least 15 days per month, still more preferably at least 20 days per month. All of these feeding frequencies, whether they be, e.g., three times per week, every other day or daily, fit under the umbrella of substantially daily provided that they promote the isoxazoline reaching and maintaining a therapeutically effective level of the isoxazoline in the feline's blood.

The term “therapeutically effective” means that the dose or blood level of isoxazoline is sufficient to control the tick infestation better than if no drug were present. The isoxazoline may be present on its own or with one or more additional active substances. Preferably it controls the tick infestation at around at least 50% better than if no drug were present, and more preferably it controls the tick infestation at about at least 90% better than if no drug were present.

In carrying out the methods of this disclosure, an effective amount of an isoxazoline is administered orally to the feline. The term “effective amount” or “therapeutically effective amount” refers to the amount needed to control the tick infestation. As those in the art will understand, this amount will vary depending upon a number of factors. These factors include, for example, the type of feline being treated and its weight and general physical condition.

Isoxazolines vary in potency. Thus, the effective amount of isoxazoline must be calculated for each particular isoxazoline used in the method according to this disclosure. In general, the effective amount for an oral daily dose of an isoxazoline will be in the range of about 12.5%-90% of the approved label dose for said isoxazoline divided by length of the dosing/retreatment interval (e.g., the dosage divided by 30 for a product administered once per month). One of skill in the art will recognize that a higher dose of, e.g., 90%-200% of the approved label dose for said isoxazoline may be selected for reasons such as, but not limited to, manufacturability, ease of testing and analysis, etc. The particular dose selected may be sufficient to raise the feline's blood concentration of said isoxazoline to a therapeutically effective level within about 7 days of substantially daily administrations, more preferably within about 5 days of substantially daily administrations, most preferably within about 3 days of substantially daily administrations.

While this disclosure describes concentrations of isoxazoline in terms of daily feeds such as kibble, it also contemplates administration using other dosage forms, such as treats or chews. It is also contemplated that the isoxazoline may be administered by itself or in a tablet, liquid, gel or other suitable form for oral administration. One of skill in the art will appreciate that the concentration of isoxazoline will vary according to the particular dosage form. For example, where the animal feed is a treat, the concentration of isoxazoline in the treat will be greater than, e.g., the concentration of isoxazoline in a kibble. For example, if the daily dose of isoxazoline based on the weight of the feline is 20 mg, then a typical 5 g treat may contain about 0.4 percent isoxazoline (by weight). Since the amount of kibble consumed in a day is more than 5 g, the percent isoxazoline in kibble will be smaller.

For example, an effective amount of mivorilaner may be a dose of from about 0.315 to about 5 mg of mivorilaner/kg of body weight of the feline. More preferably, an effective amount of mivorilaner may be a dose of from about 0.495 to about 2.25 mg of mivorilaner/kg of body weight of the feline. More commonly, the effective amount is from about 0.315 to about 1.875 mg/kg of body weight of the feline.

Animal feeds will typically contain from about 0.0015 to about 0.6 percent of mivorilaner (by weight) in the feed; preferably between about 0.003 to about 0.36 percent of mivorilaner (by weight) in the feed; most preferably between about 0.0045 to about 0.18 percent of mivorilaner component or components (by weight) in the feed.

In another example, an effective amount of lotilaner may be a dose of from about 0.124 to about 2 mg of lotilaner/kg of body weight of the feline. More preferably, an effective amount of lotilaner may be a dose of from about 0.2 to about 0.9 mg of lotilaner/kg of body weight of the feline. More commonly, the effective amount is from about 0.124 to about 0.75 mg/kg of body weight of the feline.

Animal feeds will typically contain from about 0.0006 to about 0.24 percent of lotilaner (by weight) in the feed; preferably between about 0.0012 to about 0.15 percent of lotilaner (by weight) in the feed; most preferably between about 0.003 to about 0.0075 percent of lotilaner component or components (by weight) in the feed.

In another example, an effective amount of afoxolaner may be a dose of from about 0.015 to about 0.25 mg of afoxolaner/kg of body weight of the feline. More preferably, an effective amount of afoxolaner may be a dose of from about 0.0255 to about 0.113 mg of afoxolaner/kg of body weight of the feline. More commonly, the effective amount is from about 0.015 to about 0.094 mg/kg of body weight of the feline.

Animal feeds will typically contain from about 0.000075 to about 0.24 percent of afoxolaner (by weight) in the feed; preferably between about 0.00015 to about 0.15 percent of afoxolaner (by weight) in the feed; most preferably between about 0.00045 to about 0.009 percent of afoxolaner component or components (by weight) in the feed.

In another example, an effective amount of esafoxolaner may be a dose of from about 0.0075 to about 0.12 mg of esafoxolaner/kg of body weight of the feline. More preferably, an effective amount of esafoxolaner may be a dose of from about 0.012 to about 0.0563 mg of esafoxolaner/kg of body weight of the feline. More commonly, the effective amount is from about 0.0075 to about 0.0469 mg/kg of body weight of the feline.

Animal feeds will typically contain from about 0.00003 to about 0.24 percent of esafoxolaner (by weight) in the feed; preferably between about 0.000075 to about 0.15 percent of esafoxolaner (by weight) in the feed; most preferably between about 0.00015 to about 0.0045 percent of esafoxolaner component or components (by weight) in the feed.

In another example, an effective amount of sarolaner may be a dose of from about 0.0075 to about 0.12 mg of sarolaner/kg of body weight of the feline. More preferably, an effective amount of sarolaner may be a dose of from about 0.012 to about 0.054 mg of sarolaner/kg of body weight of the feline. More commonly, the effective amount is from about 0.0075 to about 0.045 mg/kg of body weight of the feline.

Animal feeds will typically contain from about 0.00003 to about 0.24 percent of sarolaner (by weight) in the feed; preferably between about 0.00006 to about 0.15 percent of sarolaner (by weight) in the feed; most preferably between about 0.00015 to about 0.0045 percent of sarolaner component or components (by weight) in the feed.

In another example, an effective amount of fluralaner may be a dose of from about 0.063 to about 1 mg of fluralaner/kg of body weight of the feline. More preferably, an effective amount of fluralaner may be a dose of from about 0.1 to about 0.45 mg of fluralaner/kg of body weight of the feline. More commonly, the effective amount is from about 0.063 to about 0.375 mg/kg of body weight of the feline.

Animal feeds will typically contain from about 0.0003 to about 0.24 percent of fluralaner (by weight) in the feed; preferably between about 0.0006 to about 0.15 percent of fluralaner (by weight) in the feed; most preferably between about 0.0015 to about 0.045 percent of fluralaner component or components (by weight) in the feed.

In another example, an effective amount of umifoxolaner may be a dose of from about 0.0075 to about 0.12 mg of umifoxolaner/kg of body weight of the feline. More preferably, an effective amount of umifoxolaner may be a dose of from about 0.012 to about 0.056 mg of umifoxolaner/kg of body weight of the feline. More commonly, the effective amount is from about 0.0075 to about 0.0469 mg/kg of body weight of the feline.

Animal feeds will typically contain from about 0.00003 to about 0.24 percent of umifoxolaner (by weight) in the feed; preferably between about 0.000075 to about 0.15 percent of umifoxolaner (by weight) in the feed; most preferably between about 0.00015 to about 0.0045 percent of umifoxolaner component or components (by weight) in the feed.

In another example, an effective amount of tigolaner may be a dose of from about 0.0075 to about 0.12 mg of tigolaner/kg of body weight of the feline. More preferably, an effective amount of tigolaner may be a dose of from about 0.012 to about 0.0563 mg of tigolaner/kg of body weight of the feline. More commonly, the effective amount is from about 0.0075 to about 0.0469 mg/kg of body weight of the feline.

Animal feeds will typically contain from about 0.00003 to about 0.24 percent of tigolaner (by weight) in the feed; preferably between about 0.000075 to about 0.15 percent of tigolaner (by weight) in the feed; most preferably between about 0.00015 to about 0.0045 percent of tigolaner component or components (by weight) in the feed.

In another example, an effective amount of modolaner may be a dose of from about 0.015 to about 0.25 mg of modolaner/kg of body weight of the feline. More preferably, an effective amount of modolaner may be a dose of from about 0.0255 to about 0.113 mg of modolaner/kg of body weight of the feline. More commonly, the effective amount is from about 0.015 to about 0.094 mg/kg of body weight of the feline.

Animal feeds will typically contain from about 0.000075 to about 0.24 percent of modolaner (by weight) in the feed; preferably between about 0.00015 to about 0.15 percent of modolaner (by weight) in the feed; most preferably between about 0.00045 to about 0.009 percent of modolaner component or components (by weight) in the feed.

In one aspect, this disclosure relates to a method of controlling a tick infestation in a feline by administering a systemically active oral composition including isoxazoline and animal feed at least once per week, more preferably three times per week, most preferably substantially daily.

In another aspect, this disclosure relates to a systemically active oral composition that includes an isoxazoline and animal feed.

This disclosure also relates to the use of an isoxazoline for the manufacture of an animal feed or a chew for controlling a tick infestation on a feline.

This disclosure also relates to a method of controlling a tick infestation on a feline for a prolonged time, comprising orally administering daily or substantially daily doses of an effective amount of an isoxazoline to the feline. This method is especially useful for controlling ticks on a feline for a prolonged time comprising orally administering substantially daily doses of an effective amount of an isoxazoline to the feline.

An aspect of this disclosure is the oral administration of an amount of isoxazoline which is, in and of itself, ineffective or sub-optimal for controlling a tick infestation in a feline when administered as a single dose once per month, but over time with repeated consecutive administrations, as described herein, results in efficacious control of tick infestations. Ineffective or sub-optimal means that a single dosing, as well as several dosings, results in less than a 50% reduction in the tick infestation, including no, or substantially no, reduction, as compared to no drug administration at all. This reflects the chronic, rather than acute, administration aspect disclosed herein.

### EXAMPLES

The following examples illustrate the methods of this disclosure. It shall be understood that five of the following six examples are prophetic examples, as that term is understood to one of skill in the art. In other words, five of the six examples that follow are based on predicted results rather than actual experiments. However, the predicted results are based on experiments done with other animals and are believed to be sufficient for one of skill in the art to practice this disclosure.

### Example 1

Efficacy of Isoxazoline Administered per os, i.e. by mouth, to Cats for the Treatment and Control of Dermacentor variabilis.

Methods: A pool of 14 cats are to be preliminarily infested with ˜50 unfed adult D. variabilis ticks in order to produce cats that can suitably sustain a reliable infestation rate, defined as approximately 25% attached ticks being live at the end of a 48-hour period. The 12 cats with the highest live attached tick counts are to be selected for inclusion in the study. The cats are to be divided into a control group and a treatment group with 6 cats in each group.

The cats are to be housed individually during the study period and are to be fed a commercial dry cat food ration with ad libitum access to water.

Each cat in the treatment group is to receive by mouth, preferably in combination with feed, a liquid formulation of isoxazoline. The dosage of 1.125 mg/kg is to be administered to the cats on each of days 0-29.

Cats in the control group are not to receive isoxazoline or any other tick control treatment. Each cat in the treatment group is to be offered its daily ration (dry food) and the individual doses of liquid formulation are to be administered after the individual cat has eaten at least 25% of its total daily ration. After receiving the dose of isoxazoline, the cats are to be allowed to continue eating. This mimics incorporating the isoxazoline in feed. Each cat in the treatment group and the control group is to be experimentally infested with 50 adult D. variabilis ticks on test days 5, 12, 19, 28 and 35. Comb counts for live and moribund attached ticks are to be conducted on days 7, 14, 21, 30 and 37. The day 37 tick comb counts will be after an 8-day wash out period, approximately.

The expected result in such a study performed with various isoxazolines, for example, mivorilaner, is at least 60 percent reduction in attached tick counts over the control group within 7 days and a 90 percent reduction in attached tick counts over the control group within 14 days.

Using the same study method as described above, blood is to be drawn at 72, 168, 336, 504, 720 and 888 hours after the initial dose of isoxazoline is administered. The average concentration of isoxazoline in the blood can then be determined.

The average plasma concentration of isoxazoline in a feline's blood in such a study performed with various isoxazolines, for example, mivorilaner, at the 1.125 mg/kg dosage level is expected to range between about 1011 ng/mL at day 3 and about 4088 at day 21. Further, the average concentration is expected to decline to about 3025 ng/mL by day 37, approximately 8 days after the final dose is administered.

### Example 2

Efficacy of Various Doses of Isoxazoline Administered per os, i.e. by mouth, to Cats for the Treatment and Control of Dermacentor variabilis.

Methods: A pool of 46 cats are to be preliminarily infested with ˜50 unfed adult D. variabilis ticks in order to produce cats that can suitably sustain a reliable infestation rate, defined as approximately 25% of attached ticks being live at the end of a 48-hour period. The 40 cats with the highest live attached tick counts are to be selected for inclusion in the study. The cats are to be randomly assigned to a single control group and 4 treatment groups with 8 cats per treatment group.

The cats are to be housed individually during the study period and are to be fed a commercial dry cat food ration with ad libitum access to water.

Each cat in a treatment group (test groups 2-5) is to receive by mouth, preferably in combination with feed, a liquid formulation of isoxazoline. The dosage is to be administered to the cats on each of days 0-29 according to TABLE 1.

TABLE 1

| Treatment | | | Fed/Fasted |
|---|---|---|---|
| Group | Daily Dose (mg/kg) | Route | State |
| 1 (control) | 0 mg/kg | n/a | n/a |
| 2 | 0.1875 mg/kg daily for | Oral | Fed |
| | 29 consecutive days | | |
| 3 | 0.375 mg/kg daily for | Oral | Fed |
| | 29 consecutive days | | |
| 4 | 0.75 mg/kg daily for | Oral | Fed |
| | 29 consecutive days | | |
| 5 | 1.5 mg/kg daily for | Oral | Fed |
| | 29 consecutive days | | |

Cats in the control group are not to receive isoxazoline or any other tick control treatment. Each cat in the treatment group is to be offered its daily ration (dry food) and the individual doses of liquid formulation are to be administered after the individual cat has eaten at least 25% of its total daily ration. After receiving the daily dose of isoxazoline, the cats are to be allowed to continue eating. Each cat in treatment groups 3-5 and the control group is to be experimentally infested with 50 adult D. variabilis ticks on test days −2, 5, 12, 19, 28 and 35. Cats in group 2 are to be infested with 50 adult D. variabilis ticks on test days 19, 28 and 35. Comb counts for live and moribund adult ticks are to be conducted on days 2, 7, 14, 21, 30 and 37 for the control group and groups 3-5. Comb counts for live adult ticks are to be conducted on days 21, 30 and 37 for cats in group 2. The day 37 tick comb counts will be after an 8-day wash out period, approximately.

The dosage level for treatment group 2 is not expected to reliably achieve greater than 50% efficacy. The dosage level for treatment group 3 is expected to achieve greater than 50% efficacy within 7 days and may achieve 95% efficacy. The dosage levels for treatment groups 4 and 5 are expected to achieve greater than 80% efficacy within 7 days.

Using the same study method as described above, blood is to be drawn at 0, 72, 120, 168, 336, 504, 720 and 888 hours after the initial dose of isoxazoline is administered. The average concentration of isoxazoline in the blood for different dosage levels can then be determined.

The average plasma concentration of isoxazoline in a feline's blood with substantially daily dosing at the dosage level for test group 2 would be expected to range between about 203 ng/ml at 72 hours and about 630 ng/ml at 720 hours. For test group 3, the expected range would be between about 424 ng/ml at 72 hours and about 1524 ng/ml at 720 hours. For test group 4, the expected range would be between about 711 ng/ml at 72 hours and about 2811 ng/ml at 720 hours. For test group 5, the expected range would be between about 1427 ng/ml at 72 hours and about 5635 ng/ml at 720 hours. At 888 hours, approximately 8 days after the final daily dose of isoxazoline on Day 29, the expected average plasma concentration of isoxazoline in a feline's blood for test group 2 would be approximately 500 ng/ml; for test group 3, the expected average plasma concentration of isoxazoline in a feline's blood would be approximately 1000 ng/ml; for test group 4, the expected average plasma concentration of isoxazoline in a feline's blood would be approximately 2000 ng/ml; and for test group 5, the expected average plasma concentration of isoxazoline in a feline's blood would be approximately 4000 ng/ml.

### Example 3

Efficacy of Various Formulations of Isoxazoline Administered per os, i.e. by mouth, to Cats for the Treatment and Control of Dermacentor variabilis.

Methods: A pool of 36 cats are to be preliminarily infested with ˜50 unfed adult D. variabilis ticks in order to produce cats that can suitably sustain a reliable infestation rate, defined as approximately 25% of attached ticks being live at the end of a 48-hour period. The 30 cats with the highest live attached tick counts are to be selected for inclusion in the study. The cats are to be randomly assigned to a single control group and 4 treatment groups with 6 cats per treatment group.

The cats are to be housed individually during the study period and are to be fed a commercial dry cat food ration with ad libitum access to water.

Each cat in a treatment group (test groups 2-5) is to receive by mouth, preferably in combination with feed, a liquid formulation of isoxazoline. The dosage is to be administered to the cats on each of days 0-29 according to TABLE 2.

TABLE 2

| Treatment | Treatment | Anticipated Oral | |
|---|---|---|---|
| Group | Description | Dose (mg/kg) | Formulation |
| 1 | Non-treated | 0 mg/kg | n/a |
| | control | | |
| 2 | Afoxolaner | 0.075 mg/kg daily | Afoxolaner liquid |
| | | (Days 0-29) | formulation |
| 3 | Afoxolaner | 0.225 mg/kg daily | Afoxolaner liquid |
| | | (Days 0-29) | formulation |
| 4 | Fluralaner | 0.125 mg/kg daily | Fluralaner liquid |
| | | (Days 0-29) | formulation |
| 5 | Fluralaner | 0.375 mg/kg daily | Fluralaner liquid |
| | | (Days 0-29) | formulation |

Cats in the control group are not to receive isoxazoline or any other tick control treatment. Each cat in the treatment group is to be offered its daily ration (dry food) and the individual doses of liquid formulation are to be administered after the individual cat has eaten at least 25% of its total daily ration. After receiving the dose of isoxazoline, the cats are to be allowed to continue eating. This mimics incorporating the isoxazoline in feed. Each cat in the treatment group and the control group is to be experimentally infested with 50 unfed adult D. variabilis ticks on test days −2, 5, 12, 19 and 28. Comb counts for attached live and moribund adult ticks are to be conducted on days 2, 7, 14, 21 and 30.

At the dosage levels described in this example, treatment groups 3, 4 and 5 would be expected to reach greater than 95% efficacy by day 7. Treatment group 2 would be expected to reach greater than 80% efficacy by day 14 and greater than 90% efficacy by day 21.

### Example 4

Plasma Concentration of Isoxazoline in Cats when Isoxazoline is Administered in a Medicated Feed Dosed at 1.5 mg/kg of the Cat's Weight.

Methods: A pool of 30 cats are to be assigned to 5 groups of 6 cats per group by weight to minimize variation between and within the groups. Each group will be given a different feed formulation containing isoxazoline and the blood level of isoxazoline over the one month period following the single daily dose will be determined.

The cats are to be housed individually during the study period and are to have ad libitum access to water.

There is to be an initial acclimation period of at least 4 days during which cats are to be transitioned from a standard certified commercial cat feed to an unmedicated version of the daily cat feed. During the acclimation period, the cats are to be allowed 15 minutes/day to consume the feed.

Each medicated feed formulation should be able to deliver approximately 14.1 g/kg of daily feed containing isoxazoline. The amount of medicated feed for each cat is to be determined according to the most recent body weight of the cat prior to the first day (Day 0) of the study. Each medicated feed is to be provided for 15 minutes daily from Day 0 up to and including Day 30. Any uneaten medicated feed remaining after each daily 15 minute timed offering period is to be removed, weighed and the amount recorded. An amount of unmedicated feed equaling the amount of uneaten medicated feed is to be provided ten hours later, at the first blood sampling time.

Blood samples are to be taken at the following times: 0 hr (at the time the medicated feed is first provided), 0.25 hr, 0.5 hr, 1 hr, 3 hr, 6 hr, 10 hr, 1 day, 2 days, 4 days, 6 days, 9 days, 13 days, 20 days, 27 days and 31 days after the medicated feed is first provided on Day 0.

The mean plasma concentrations in such a study performed with various isoxazolines, for example, mivorilaner, would be expected to produce plasma concentrations consistent with those achieved in Examples 1 and 2, above, irrespective of the different formulations of the feed.

### Example 5

Efficacy of Isoxazoline when Isoxazoline is Administered in a Medicated Feed for the Treatment and Control of Dermacentor variabilis.

Methods: A pool of 24 cats are to be preliminarily infested with ˜50 unfed adult D. variabilis ticks in order to produce cats that can suitably sustain a reliable infestation rate, defined as approximately 25% of attached ticks being live at the end of a 48-hour period. The 18 cats with the highest live attached tick counts are to be randomly assigned to a single control group (Group 1) and 2 different medicated feed treatment groups (Groups 2 and 3) with 6 cats per group. The remaining 6 cats are to be assigned to a final medicated feed treatment group (Group 4).

The cats are to be housed individually during the study period and are to have ad libitum access to water.

Each cat in a treatment group (Groups 2-4) is to receive a medicated daily feed from study days 0-29. Each medicated daily feed (Groups 2-4) is to be offered to the cats for 1 hour on each of days 0-29 according to test groups in TABLE 3.

TABLE 3

| | | Anticipated | |
|---|---|---|---|
| Treatment | # of | Daily Dose | Infestation |
| Group | Cats | (mg/kg) | with Ticks |
| 1 | 6 | 0 | Yes |
| 2 | 6 | 1 | Yes |
| 3 | 6 | 1 | Yes |
| 4 | 6 | 3 | No |

Cats in the control group are not to receive isoxazoline or any other tick control treatment. Each cat in treatment groups 2 and 3 and the control group is to be experimentally infested with 50 unfed adult D. variabilis ticks on test days −2, 4, 12 and 28 during the treatment phase and on day 36 during the wash out period after the final feeding with the medicated daily feed. Comb counts for attached live and moribund adult ticks are to be conducted on days 3, 6, 14, 30 and 38.

At the dosage levels in a study according to this example performed with various isoxazolines, for example, mivorilaner, treatment groups 2 and 3 would be expected to reach greater than 95% efficacy by day 14.

Using the same study method as described above, blood is to be drawn at day 0 (day of the first administration of medicated feed) and days 2, 4, 7, 10, 14, 21 and 30 after the initial dose of isoxazoline is administered on day 0. The average concentration of isoxazoline in the blood for different dosage levels can then be determined.

The plasma concentrations of isoxazoline in such a study performed with various isoxazolines, for example, mivorilaner, are expected to be consistent between treatment groups 2 and 3, regardless of the difference in feed formulations. The plasma concentrations of isoxazoline for cats in treatment group 4 are expected to be approximately 2.5-4 times higher than the plasma concentrations of cats in treatment groups 2 and 3.

### Example 6

Efficacy of Isoxazoline when Isoxazoline is Administered in a Medicated Feed for the Treatment and Control of Dermacentor variabilis.

Methods: A pool of cats are to be preliminarily infested with ˜100 unfed adult C. felis fleas in order to produce cats that can suitably sustain a reliable infestation rate, defined as approximately 50% retention of live fleas at the end of a 48-hour period. The cats with the highest live flea counts are to be randomly assigned to a single control group (Group 1) and 2 medicated feed treatment groups with 4 cats per group. The cats in groups 2 and 3 will receive Mivorilaner.

The cats are to be housed individually during the study period and are to have ad libitum access to water.

Each cat in a treatment group (Groups 2 and 3) is to receive a medicated daily feed from study days 0-29 according to TABLE 4.

TABLE 4

| | | Anticipated | |
|---|---|---|---|
| Treatment | # of | Daily Dose | Infestation |
| Group | Cats | (mg/kg) | with Ticks |
| 1 | 4 | 0 | Yes |
| 2 | 4 | 0.75 | Yes |
| 3 | 4 | 1.5 | Yes |

The cats are to be fasted overnight prior to each daily treatment. The daily dose volume of Mivorilaner using a liquid formulation is to be mixed into a small portion (approximately 25% of the cat's daily dietary needs) of wet canned cat food. Approximately 4 hours after the medicated feed has been initially offered, the remaining medicated feed, if any, will be mixed with the 75% unmedicated daily feed for each cat. The resulting feed offering may be consumed by the cats until the feed bowls are removed for the daily overnight fast.

Cats in the control group are not to receive isoxazoline or any other tick control treatment. Each cat in treatment groups 2 and 3 and the control group is to be experimentally infested with 50 unfed adult D. variabilis ticks on test days −2, 14, 25 and 28 during the treatment phase and on day 35 with a wash out period of no daily medicated dosing starting on day 30 (simulating an approximately 8-day washout period with the tick count on day 37). Comb counts for attached live and moribund adult ticks are to be conducted on days 2, 16, 27, 30 and 37.

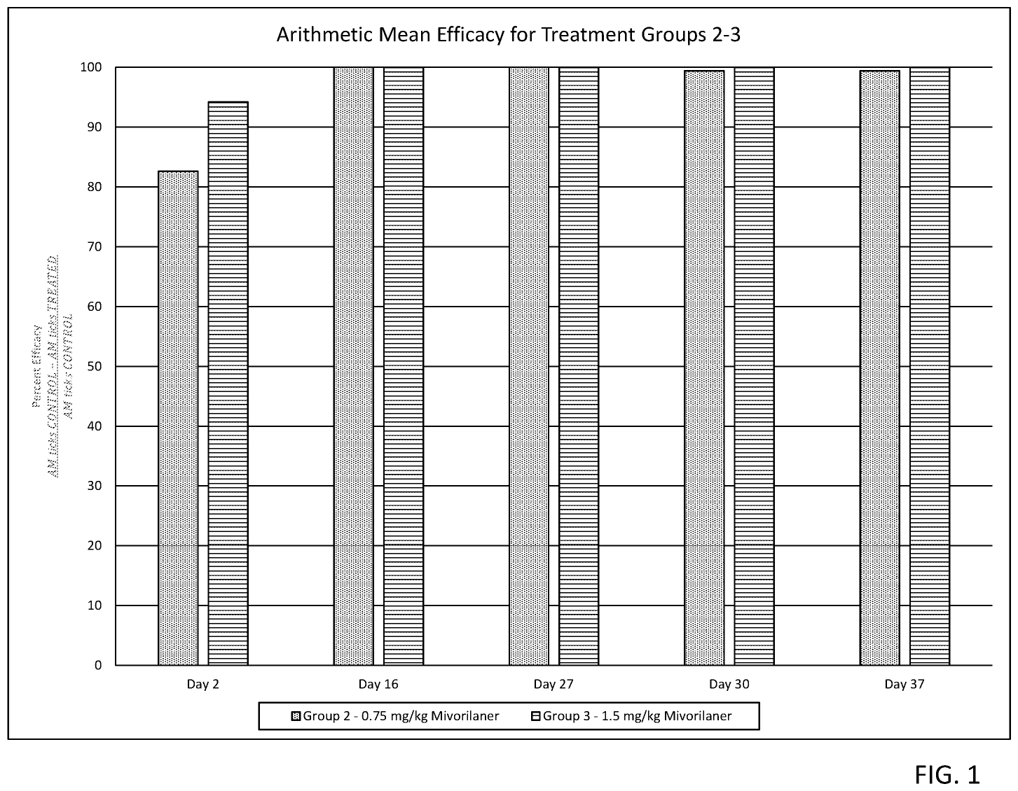

Results: Percent reduction in attached live and moribund tick counts for treatment groups 2 and 3 according to this example are shown in FIG. 1. Both treatment groups reach 100% efficacy within 16 days.

While this invention has been described as having an exemplary design, the present invention may be further modified within the spirit and scope of this disclosure. This application is therefore intended to cover any variations, uses, or adaptations of the invention using its general principles.

What is claimed is:

1. A method of controlling a tick infestation in a feline in need thereof, comprising orally administering to said feline an effective amount of an isoxazoline for an effective time at a frequency of at least four times per month.

2. The method of claim 1, wherein said feline is a domestic cat.

3. The method of any of claim 1, wherein said isoxazoline is mivorilaner, or a salt thereof.

4. The method of claim 3, wherein said mivorilaner is provided in a feed in an amount selected from the group consisting of between about 0.0015 to about 0.6 percent by weight of the feed and between about 0.003 to about 0.36 percent by weight of the feed.

5. The method of claim 3, wherein said mivorilaner is administered to said feline in an amount selected from the group consisting of between about 0.315 mg/kg and about 5 mg/kg of body weight of said feline and between about 0.495 mg/kg and about 2.25 mg/kg of body weight of said feline.

6. The method of claim 3, wherein said administration provides a concentration of mivorilaner of more than about 600 ng/mL and less than about 18,000 ng/mL in said feline's blood for a period of time selected from the group consisting of at least 30 days and at least 365 days.

7. The method of claim 1, wherein said isoxazoline is at least one isoxazoline selected from the group consisting of fluralaner, sarolaner, afloxolaner, and lotilaner or a salt thereof.

8. The method of claim 1, wherein said isoxazoline is administered as a component of a feed.

9. The method of claim 1, wherein said frequency is selected from the group consisting of at least 3 times per week, substantially daily and daily.

10. The method of claim 1, wherein said effective time comprises administering the isoxazoline for a time period selected from the group consisting of at least one week and at least two weeks.

11. The method of claim 1, wherein said administration provides a therapeutically effective level of isoxazoline in said feline's blood within a time period selected from the group consisting of one week of the first administration of said isoxazoline and two days of the first administration of said isoxazoline.

12. The method of claim 1, wherein said administration provides a therapeutically effective level of isoxazoline in said feline's blood for a time period selected from the group consisting of at least 30 days, at least 60 days, at least 90 days, at least 180 days and at least 365 days.

13. The method of claim 1, wherein said isoxazoline is administered for a period of time selected from the group consisting of at least 15 out of 30 days and at least 20 out of 30 days.

14. The method of claim 1, further comprising discontinuing the administration of the isoxazoline for a period of time selected from the group consisting of at least 3 days and at least 7 days, wherein the feline's blood concentration of isoxazoline is maintained at a therapeutically effective level.

15. The method of claim 14, further comprising resuming the administration of the isoxazoline after the discontinuing of the administration of the isoxazoline and thereby maintaining the feline's blood concentration of isoxazoline at the therapeutically effective level.

16. A feed or chew for controlling ticks in a feline, said feed or chew comprising an effective amount of an isoxazoline to control a tick infestation when administered to said feline for an effective time at a frequency of at least four times per month.

17. The feed or chew according to claim 16, wherein the isoxazoline is at least one isooxazoline selected from the group consisting of mivorilaner, fluralaner, sarolaner, afloxolaner, and lotilaner or a salt thereof.

18. The feed or chew according to claim 17, wherein said mivorilaner is provided in a feed in an amount selected from the group consisting of between about 0.0015 to about 0.6 percent by weight of the feed and between about 0.003 to about 0.36 percent by weight of the feed.

19. The feed or chew according to claim 17, wherein said mivorilaner is administered to said feline in an amount selected from the group consisting of between about 0.315 mg/kg and about 5 mg/kg of body weight of said feline and between about 0.495 mg/kg and about 2.25 mg/kg of body weight of said feline.

20. The feed or chew according to claim 17, wherein said administration provides a concentration of mivorilaner of more than about 600 ng/mL and less than about 18,000 ng/mL in said feline's blood for a period of time selected from the group consisting of at least 30 days and at least 365 days.
